(19) United States
(12) Patent Application Publication
(10) Pub. No.: US 2020/0171221 A1
(43) Pub. Date: Jun. 4, 2020

(54) Negative Pressure Sealing Drainage System for Surgical Treatment of Deep Cavity Wound

(71) Applicant: WUHAN VSD MEDICAL SCIENCE & TECHNOLOGY CO., LTD., Wuhan, Hubei (CN)

(72) Inventors: Yuming ZHU, Wuhan (CN); Xinting LIU, Wuhan (CN); Hongxia GUAN, Wuhan (CN); Jiuhong SONG, Wuhan (CN)

(73) Assignee: Wuhan VSD Medical Science & Technology Co., Ltd., Wuhan, Hubei (CN)

(21) Appl. No.: 16/310,077

(22) PCT Filed: May 24, 2017
(86) PCT No.: PCT/CN2017/085628

§ 371 Date: Dec. 14, 2018

(30) Foreign Application Priority Data
Jun. 15, 2016 (CN) 201610424612.2

(51) Int. Cl. A61M 1/00

(52) U.S. Cl. CPC A61M 1/0088; A61M 2205/3344; A61M 1/0001; A61M 1/0023

(57) ABSTRACT

A negative pressure sealing drainage system for surgical treatment of deep cavity wound includes: a columnar porous foam drainage piece, a wave-form sealing cover which is extendable and retractable, a medical film, a drainage tube, a fluid storage container, and a negative pressure source. The columnar porous foam drainage piece is a hydrophilic foam, and is enclosed within the wave-form sealing cover. One end of the drainage tube is inserted into an upper portion of the columnar porous foam drainage piece. A flap provided along a circumference of the bottom-most end of the wave-form sealing cover adheres to skin surrounding a deep cavity wound. An upper end of the wave-form sealing cover is provided with a tube-shaped connection opening sealingly connected to the drainage tube communicating with a connection tube via a straight-tube fitting and inserted into the fluid storage container. The fluid storage container communicates with the negative pressure source via a connection tube.

The present disclosure claims the priority to the Chinese patent application with the filing number 201610424612.2, entitled “Negative Pressure Sealing Drainage System for Surgical Treatment of Deep Cavity Wound”, filed on Jun. 15, 2016 with Chinese Patent Office, the contents of which are incorporated in the present disclosure by reference in its entirety.

### TECHNICAL FIELD

The present disclosure relates to the technical field of medical devices, particularly to a negative pressure (vacuum) sealing drainage system for surgical treatment of deep cavity wound.

### BACKGROUND ART

Currently for superficial wounds, replacement of dressing is generally employed for treatment in clinical, which has a long period of treatment and poor therapeutic effect, and gauze is easy to be soaked by exudate, causing pollution and reinfection, and the gauze needs to be replaced frequently.

For patients with a deep cavity wound and a relatively small section of a hydrops distribution area, common foam currently used can hardly go deep to a root portion of the wound, with modest effect of being fitted to the wound. With gradual recovery of the deep cavity wound of the patient, a wound area is reduced continuously, and medical personnel need to replace a foam material with a size corresponding to the wound area multiple times, and again paste a film to seal the deep cavity wound. High frequency of operation and frequent replacement of a foam soft cushion increase the probability of secondary infection of the patient's wound.

### SUMMARY

An object of the present disclosure is to provide a negative pressure sealing drainage system for surgical treatment of deep cavity wound, to overcome shortcomings in the above background art. In a drainage process, depending on an extendable and retractable effect of a wave-form extendable and retractable sealing cover (i.e. a wave-form sealing cover which is extendable and retractable) itself, the foam can be pulled out from the deep cavity wound through several operations, ensuring airtightness of the system, avoiding film pasting for a second time, reducing difficulty of medical care task, facilitating and simplifying the operation, being capable of avoiding replacement of the foam material and dispensed with multiple times of film pasting, which promotes quick healing of the deep cavity wound. Effective negative pressure sealing drainage is provided to patients with a relatively deep wound and a relatively small section of a hydrops distribution area.

Embodiments of the present disclosure are implemented as follows:

An embodiment of the present disclosure provides a negative pressure sealing drainage system for surgical treatment of deep cavity wound, which includes a porous foam, a medical film (medical pasting film), a drainage tube, a fluid storage container, and a negative pressure source, wherein the porous foam is a columnar porous foam drainage piece, one end of the drainage tube is inserted into an upper end of the columnar porous foam drainage piece, the columnar porous foam drainage piece is covered by a wave-form sealing cover which is extendable and retractable, an upper end of the wave-form sealing cover is provided with a tube-shape connection port hermetically connected to the drainage tube, at a lower end of the wave-form sealing cover which is extendable and retractable, there is an outer-edge flap, and the medical film is covered on the outer-edge flap.

Optionally, the columnar porous foam drainage piece has a cross-sectional area of 0.8 cm²˜3 cm².

Optionally, the columnar porous foam drainage piece has a length of 10 cm˜28 cm.

Optionally, the columnar porous foam drainage piece has foam pores, and the foam pores have a diameter of 1˜2 mm.

Optionally, a material of the columnar porous foam drainage piece is hydrophilic foam.

Optionally, a length of the wave-form sealing cover in a natural state is less than a length of the columnar porous foam drainage piece by 20 mm˜50 mm.

Optionally, the outer-edge flap at a lower end of the wave-form sealing cover which is extendable and retractable has a radial width of 10 mm˜20 mm.

Optionally, the wave-form sealing cover which is extendable and retractable is a wave-form adhesive-film sealing cover which is extendable and retractable.

Optionally, the wave-form adhesive-film sealing cover has a wall thickness of 0.8˜1 mm.

Optionally, a gap between an inner chamber of the wave-form adhesive-film sealing cover in a natural state and the columnar porous foam drainage piece is 2 mm˜8 mm.

Optionally, the tube-shape connection port at an upper end of the wave-form adhesive-film sealing cover which is extendable and retractable has a wall thickness of 2 mm˜4 mm.

Optionally, the wave-form sealing cover is a plastic corrugated-tube sealing cover which is extendable and retractable.

Optionally, the plastic corrugated-tube sealing cover has a wall thickness of 0.3 mm˜0.6 mm.

Optionally, a minimum gap between an inner chamber of the plastic corrugated-tube sealing cover in a natural state and the columnar porous foam drainage piece is 2 mm˜8 mm.

Optionally, the tube-shape connection port at an upper end of the plastic corrugated-tube sealing cover has a wall thickness of 1 mm˜3 mm.

Optionally, the drainage tube includes a connection tube segment and a drainage segment interconnected, the connection tube segment is connected to the fluid storage container, an end of the drainage segment away from the connection tube segment is connected to the columnar porous foam drainage piece, the connection tube segment has a hollow inner chamber, the drainage segment has an inner chamber, and the drainage segment is provided with a notch communicating with the inner chamber.

Optionally, the drainage tube has an outer diameter of 6 mm, and an inner diameter of 4 mm.

Optionally, the notch is a groove provided on a tube wall of the drainage segment.

Optionally, the number of the grooves is four, and the drainage segment is partitioned by the grooves into four portions.

Optionally, the inner chamber of the drainage segment is provided with a cross supporting rib for supporting the tube wall of the drainage segment, and four ends of the cross supporting rib are connected to the four portions of the drainage segment, respectively.

Optionally, the drainage tube is a diameter-variable tube, and the diameter-variable segment of the diameter-variable tube has a diameter of 12 mm-24 mm.

Optionally, the connection tube segment has an outer diameter of 6 mm, and an inner diameter of 4 mm.

Optionally, the notch is a side hole provided on the tube wall of the drainage segment.

Optionally, the inner chamber of the drainage segment is provided with a sawtooth-shape supporting rib for supporting the tube wall of the drainage segment, the sawtooth-shape supporting rib is located on an upper wall of the inner chamber, and tooth tips of the sawtooth-shape supporting rib are oriented towards a tube axis of the diameter-variable tube.

Optionally, in the connection tube segment, one flushing tube is inserted into the hollow inner chamber, and when approaching the drainage segment, the flushing tube is drawn out of the hollow inner chamber, and the flushing tube drawn out of the hollow inner chamber runs parallel to the drainage segment.

Compared with the prior art, embodiments of the present disclosure have following beneficial effects:

1. Alternative to the conventional treatment of replacing dressings, the negative pressure drainage is used in the whole system, then patients' pains can be reduced, and the degree of satisfaction of family members is improved. Compared with dotted drainage with a drainage tube, large-area growth of granulation tissues of the wound can be improved at the same time of drainage, and the patients' hospital stays can be shortened.

2. The present disclosure effectively solves the sealing problem of the columnar porous foam piece. In the drainage process, depending on an extendable and retractable effect of the wave-form sealing cover itself, the foam can be pulled out from the deep cavity wound through several operations, ensuring airtightness of the system, avoiding film pasting for a second time, reducing difficulty of medical care task, and facilitating and simplifying the operation.

3. An arc-shape design is employed for the porous foam. Compared with the conventional cubic drainage foam, a dimension of the porous foam is designed in a more ergonomic manner. When the foam material is finally pulled out, rather than an unhealed periwound with a corner, but a periwound corresponding to a section of the arc-shape foam will be formed on a body surface, and after being sutured, the wound can be timely closed.

4. The drainage tube laterally opened with grooves has a large contact area with the porous foam, then the negative pressure is transmitted more quickly, and the drainage effect is better. The drainage segment of the diameter-variable drainage tube has a larger section, which is applicable to postoperative patients with relatively more interstitial fluid in the wound cavity or sinus tract and abdominal cavity drainage after abdominal cavity operations and so on.

The whole system provides an effective antibacterial environment, avoids secondary infection to the wound, reduces toxic absorption of the body, and decreases wound infection and bleeding, so as to accelerate wound healing.

### BRIEF DESCRIPTION OF DRAWINGS

FIG. 1 is a schematic diagram of a negative pressure sealing drainage system for surgical treatment of deep cavity wound provided in an embodiment of the present disclosure;

FIG. 2 is a structural schematic diagram of a columnar porous foam drainage piece;

FIG. 3 is a structural schematic diagram of a drainage tube containing grooves;

FIG. 4 is a sectional view of a drainage segment of the drainage tube containing grooves;

FIG. 5 is a structural schematic diagram of the drainage tube containing side holes;

FIG. 6 is a structural schematic diagram of a diameter-variable drainage tube;

FIG. 7 is a sectional view of a drainage segment of the diameter-variable drainage tube;

FIG. 8 is a structural schematic diagram of a wave-form adhesive-film sealing cover which is extendable and retractable;

FIG. 9 is a structural schematic diagram of a plastic corrugated-tube sealing cover which is extendable and retractable;

FIG. 10 is a schematic diagram of the negative pressure sealing drainage system for surgical treatment of deep cavity wound in a use state; and

FIG. 11 is a partially enlarged schematic diagram of a tangent plane of a negative pressure sealing drainage area shown in FIG. 10.

In the drawings: 1—columnar porous foam drainage piece; 2—wave-form sealing cover which is extendable and retractable; 2a—tube-shape connection port; 2b—outer-edge flap; 3—medical film; 4—drainage tube; 4a—drainage segment; 4b—connection tube segment; 4c—groove; 4d—side hole; 4e—sawtooth-shape supporting rib; 4f—cross supporting rib; 5—straight-tube fitting; 6—connecting tube; 6a—first connection tube; 6b—second connection tube; 7—fluid storage container; 8—negative pressure source; 9—flushing tube.

### DETAILED DESCRIPTION OF EMBODIMENTS

The implementation situation of the present disclosure is described in detail below in combination with accompanying drawings, while they do not constitute limitation to the disclosure, but merely as examples illustrated. Meanwhile, advantages of the present disclosure will become much clearer and liable to be understood according to the description.

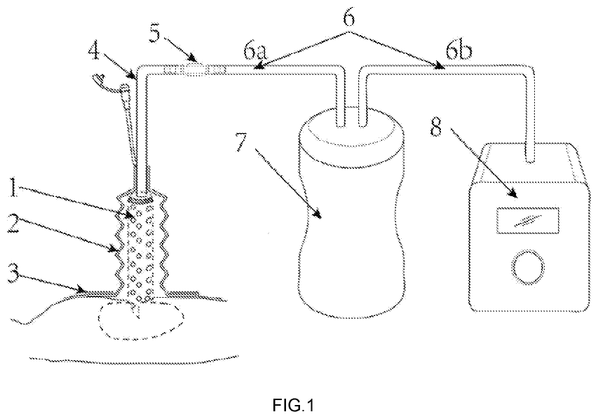

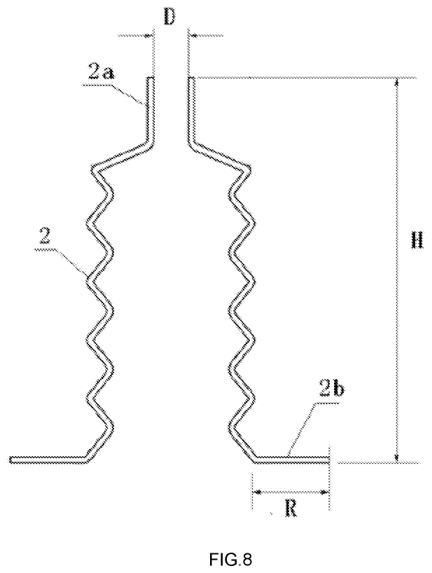

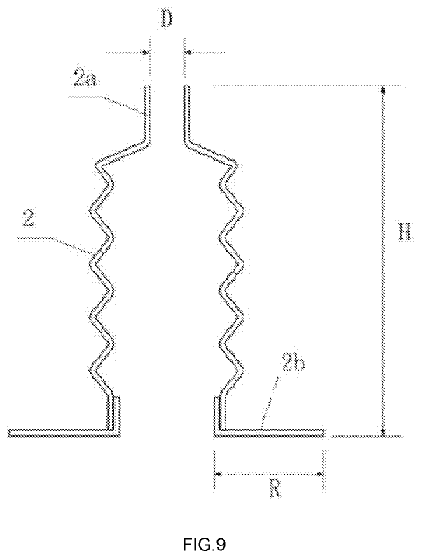

As in FIG. 1, FIG. 8, and FIG. 9: a negative pressure sealing drainage system for surgical treatment of deep cavity wound in the present disclosure includes a porous foam, a medical film 3, a drainage tube 4, a fluid storage container 7, and a negative pressure source 8. The porous foam is a columnar porous foam drainage piece 1. One end of the drainage tube 4 is inserted into an upper end of the columnar porous foam drainage piece 1. The columnar porous foam drainage piece 1 is covered by a wave-form sealing cover 2 which is extendable and retractable. At an upper end of the wave-form sealing cover 2, there is a tube-shape connection port 2a hermetically connected to the drainage tube 4. At a lower end of the wave-form sealing cover 2, there is an outer-edge flap 2b, and the medical film 3 is covered on the outer-edge flap 2b. The drainage tube 4 communicates with a first connection tube 6a through a straight-tube fitting 5, and is connected into the fluid storage container 7. The second connection tube 6b connects the fluid storage container 7 to the negative pressure source 8.

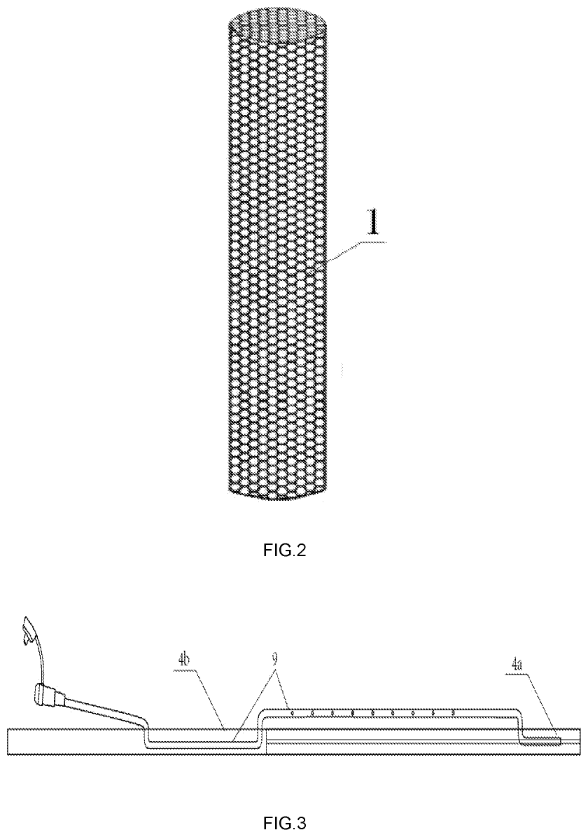

FIG. 2 is a structural schematic diagram of the columnar porous foam drainage piece.

The columnar porous foam drainage piece 1 has a sectional area of 0.8˜3 cm², a length of 10˜28 cm, and a foam pore diameter of 1˜2 mm, and a material of the columnar porous foam drainage piece 1 is hydrophilic foam.

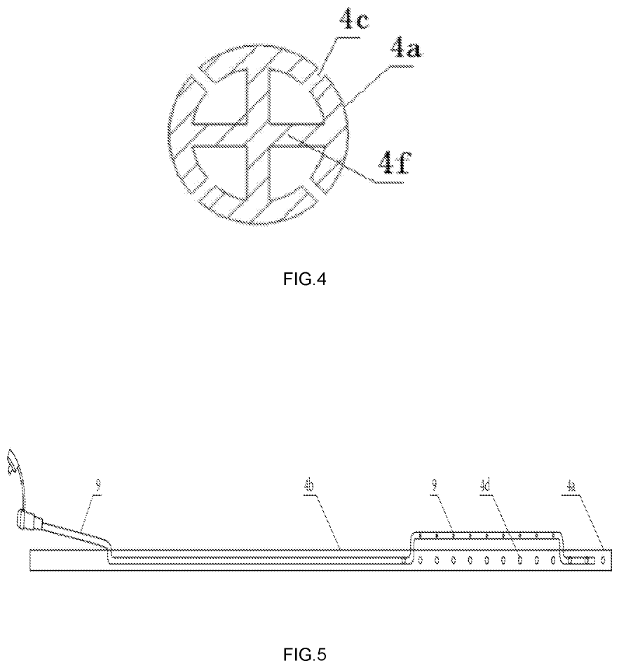

FIG. 3 and FIG. 4 are structural schematic diagrams of the drainage tube 4 containing grooves.

The drainage tube 4 has an outer diameter of 6 mm, and an inner diameter of 4 mm, and includes a connection tube segment 4b and a drainage segment 4a. On a tube wall of the drainage segment 4a thereof, there are four grooves 4c communicating with an inner chamber, respectively. The connection tube segment 4b has a hollow inner chamber. At the connection tube segment 4b, one flushing tube 9 is inserted into the hollow inner chamber, and when approaching the drainage segment 4a, the flushing tube 9 is drawn out of the hollow inner chamber, and runs parallel to the drainage segment 4a.

As in FIG. 4: the grooves containing drainage segment 4a of the drainage tube 4 is a four-chamber drainage tube, and there is a cross supporting rib 4f in the inner chamber of the drainage segment 4a, forming multiple chambers, and preventing the drainage tube 4 from being deflated by negative pressure in a process of being sucked by a negative pressure.

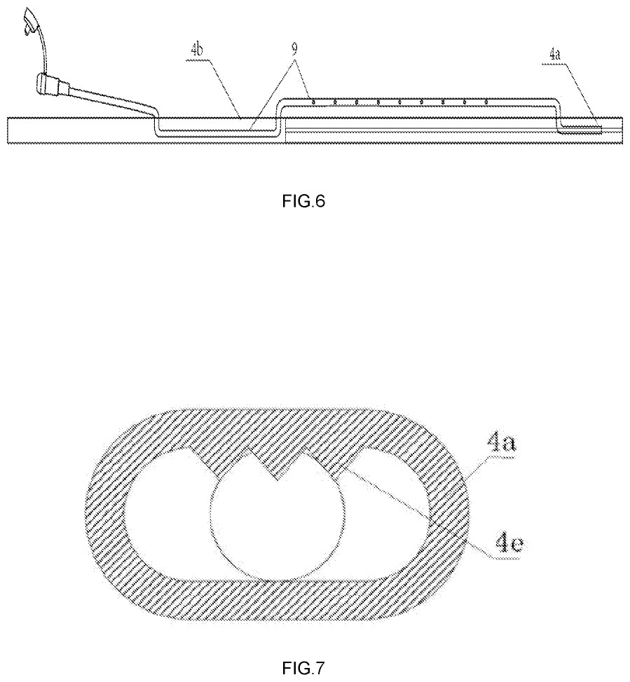

FIG. 5 is a structural schematic diagram of the drainage tube 4 containing side holes.

The drainage tube 4 containing side holes has an outer diameter of 6 mm, and an inner diameter of 4 mm. On the tube wall of the drainage segment 4a thereof, there are side holes 4d communicating with the inner chamber. The connection tube segment 4b has a hollow inner chamber. At the connection tube segment 4b, one flushing tube 9 is inserted into the hollow inner chamber, and when approaching the drainage segment 4a, the flushing tube 9 is drawn out of the hollow inner chamber, and runs parallel to the drainage segment 4a.

FIG. 6 is a structural schematic diagram of the drainage tube 4 with a variable diameter.

The drainage segment 4a of the drainage tube 4 with a variable diameter has a diameter of 12 mm-24 mm. On the tube wall of the drainage segment 4a, there are side holes 4d communicating with the inner chamber. The connection tube segment 4b has a hollow inner chamber, with an outer diameter of 6 mm, and an inner diameter of 4 mm. At the connection tube segment 4b, one flushing tube 9 is inserted into the hollow inner chamber, and when approaching the drainage segment 4a, the flushing tube 9 is drawn out of the hollow inner chamber, and runs parallel to the drainage segment 4a.

FIG. 7 is a structural schematic diagram of a section of the drainage tube 4 with a variable diameter.

On an upper wall of the inner chamber of the drainage segment 4a of the drainage tube 4 with a variable diameter, there is a sawtooth-shape supporting rib 4e, preventing the drainage tube 4 from being deflated by negative pressure in a process of being sucked by a negative pressure.

FIG. 8 is a structural schematic diagram of a wave-form adhesive-film sealing cover which is extendable and retractable.

When the wave-form sealing cover 2 is the wave-form adhesive-film sealing cover which is extendable and retractable, the wave-form adhesive-film sealing cover has a wall thickness of 0.8˜1 mm, a length H of the wave-form adhesive-film sealing cover in a natural state is less than a length of the columnar porous foam drainage piece 1 by 20˜50 mm, a gap between an inner chamber of the wave-form adhesive-film sealing cover in a natural state and the columnar porous foam drainage piece 1 is 2 mm<S<8 mm, a tube-shape connection port 2a at an upper end of the wave-form adhesive-film sealing cover has a wall thickness of 2˜4 mm, and a diameter D matched with that of the drainage tube 4, and an outer-edge flap 2b at a lower end of the wave-form adhesive-film sealing cover has a radial width R of 10˜20 mm.

FIG. 9 is a structural schematic diagram of a plastic corrugated-tube sealing cover which is extendable and retractable.

When the wave-form sealing cover 2 is a plastic corrugated-tube sealing cover which is extendable and retractable, the plastic corrugated-tube sealing cover has a wall thickness of 0.3˜0.6 mm, a length of the plastic corrugated-tube sealing cover in a natural state is less than a length of the columnar porous foam drainage piece 1 by 20˜50 mm, a minimum gap between an inner chamber of the plastic corrugated-tube sealing cover in a natural state and the columnar porous foam drainage piece 1 is 2 mm˜8 mm, a tube-shape connection port 2a at an upper end of the plastic corrugated-tube sealing cover has a wall thickness of 1˜3 mm, and an outer-edge flap 2b at a lower end of the plastic corrugated-tube sealing cover has a radial width of 10˜20 mm.

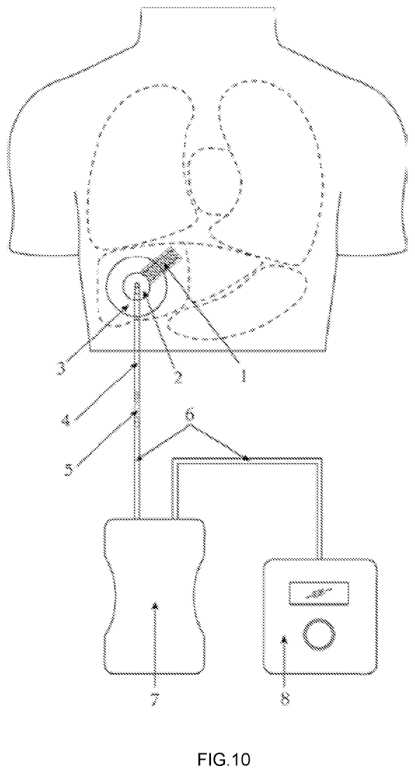

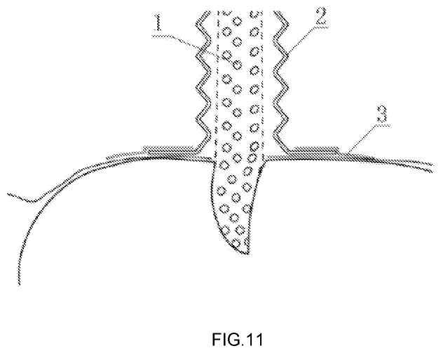

FIG. 10 is a schematic diagram of negative pressure drainage system for surgical treatment of deep cavity wound in a use state, and FIG. 11 is an enlarged diagram of a tangent plane of a sealing drainage area when the negative pressure sealing drainage system is used in a surgical treatment of deep cavity wound.

In use, the columnar porous foam drainage piece 1 is placed in a deep cavity wound to-be-drained area, then is connected to the wave-form sealing cover 2 through the drainage tube 4, and is closely fitted to skin surrounding the deep cavity wound through the medical film 3, forming a hermetic environment. The negative pressure source is turned on, to suck exudate of the deep cavity wound out of body.

A working process: according to analysis of the negative pressure drainage process for the deep cavity wound, with growth of granulation tissues, the wound heals gradually, and an inner volume of a chamber passage is also correspondingly reduced, therefore, ensuring that the foam material can be pulled out through several operations without destroying airtightness of the system is a main function of the present disclosure. An operating process thereof is as follows.

Firstly, the deep cavity wound is detected for size and depth, and a suitable columnar porous foam drainage piece is chosen according to the size and depth thereof, and trimmed. Use requirement: the columnar porous foam drainage piece should be, in length, longer than the deep cavity wound by 3-5 cm.

Then, the columnar porous foam drainage piece covered by a thread-type sealing cover which is extendable and retractable, together with the drainage tube, is placed into the deep cavity wound, and the thread-type sealing cover is fitted to skin surrounding the wound with the medical film, forming a closed system. Requirement: the porous foam need to penetrate into a deepest position of the deep cavity wound, with an object thereof being sucking out interstitial fluid in the deepest position of the deep cavity wound through the negative pressure source, so as to achieve an anaerobic environment where microorganism propagation is undermined, to prevent microorganisms from continuing to propagate in the wound cavity.

Finally, the negative pressure source is turned on, and the porous foam is adsorbed inside the deep cavity wound through negative pressure attraction, to fill the wound cavity. Healing capability of the patient's wound cavity is observed, to determine time point of each pull-out and number of times of pull-out. Requirement: in order to avoid occurrence of a situation of slippage of the porous foam in the pull-out process, in operation, operating forceps are needed to simultaneously clamp one end of the thread-type sealing cover and the columnar porous foam drainage piece, and then pull out a part. Taking full advantage of the extendable and retractable function of the thread-type sealing cover which is extendable and retractable, the porous foam is under hermetic protection.

Working principle: in the present disclosure, the columnar porous foam drainage piece is utilized as a medium between the negative pressure drainage tube and the deep cavity wound, and the negative pressure is transmitted through the drainage tube to the columnar porous foam drainage piece, and is uniformly distributed on a surface of the columnar porous foam drainage piece. Due to high plasticity of the columnar porous foam drainage piece, the negative pressure can reach every point of a drained area of the deep cavity wound, forming all-around drainage. Under the effect of the negative pressure, the interstitial fluid drained out enters the drainage tube through pores of the columnar porous foam drainage piece, and is quickly absorbed into the fluid storage container. The system is isolated from outside by sealing the wound, preventing pollution and cross infection, and ensuring continuous existence of the negative pressure. The continuous negative pressure enables exudate of the wound cavity to be immediately absorbed away, so as to effectively maintain the wound clean and inhibit bacteria growth.

### Embodiments

Referring to FIG. 1, a negative pressure sealing drainage system for surgical treatment of deep cavity wound shown in the figure includes a columnar porous foam drainage piece 1, a medical film 3, a drainage tube 4, a fluid storage container 7, and a negative pressure source 8. The drainage tube 4 has one end inserted into an upper end of the columnar porous foam drainage piece 1, and the other end communicating with a first connection tube 6a through a straight-tube fitting 5. An end of the first connection tube 6a away from the straight-tube fitting 5 is connected into the fluid storage container 7. The second connection tube 6b also has one end connected into the fluid storage container 7, and the other end connected to the negative pressure source 8.

The columnar porous foam drainage piece 1 is covered by a wave-form sealing cover which is extendable and retractable 2, and an upper end of the wave-form sealing cover which is extendable and retractable 2 is hermetically connected to the drainage tube 4. A lower end of the wave-form sealing cover which is extendable and retractable 2 is connected with an outer-edge flap 2b. A lower end of the columnar porous foam drainage piece 1 is placed in a deep cavity wound to-be-drained area. The outer-edge flap 2b is fitted to skin surrounding the deep cavity wound, and the medical film is covered on the outer-edge flap 2b, forming a hermetic environment.

An end of a flushing tube 9 is inserted into the drainage tube 4.

Referring to FIG. 2, the columnar porous foam drainage piece 1 is in a cylindrical shape, which has dense foam pores on its surface.

Referring to FIG. 3, the drainage tube 4 includes a connection tube segment 4b and a drainage segment 4a. The drainage segment 4a has an inner chamber, and the connection tube segment 4b has a hollow inner chamber. The flushing tube 9 has an end inserted into the hollow inner chamber, and is drawn out of the hollow inner chamber at a place approaching the drainage segment 4a. The flushing tube 9 drawn out of the hollow inner chamber runs parallel to the drainage segment 4a, and is inserted back into the inner chamber of the drainage segment 4a at a place close to a terminal end of the drainage segment 4a. The flushing tube 9 has via holes on a surface.

Referring to FIG. 4, a tube wall of the drainage segment 4a is opened with four grooves 4c, respectively, wherein the four grooves 4c are opened at equal intervals, all of the four grooves 4c communicate with the inner chamber of the drainage segment 4a, and the drainage segment 4a is partitioned by the four grooves 4c into four portions. There is a cross supporting rib in the inner chamber of the drainage segment 4a, and four ends of the cross supporting rib away from a cross center are connected to the above four portions, respectively.

Referring to FIG. 5, a tube wall of the drainage segment 4a of the drainage tube 4 is opened with side holes, and the side holes communicate with the inner chamber. The connection tube segment 4b has a hollow inner chamber. At the connection tube segment 4b, one flushing tube 9 is inserted into the hollow inner chamber, and when approaching the drainage segment 4a, the flushing tube is drawn out of the hollow inner chamber, parallel to the drainage segment 4a, and inserted back into the inner chamber of the drainage segment 4a at a place close to the terminal end of the drainage segment 4a. The flushing tube 9 has via holes on a surface.

Referring to FIG. 6, the drainage tube 7 is a diameter-variable tube, and the drainage segment 4a of the diameter-variable tube has a diameter greater than that of the connection tube segment 4b. The tube wall of the drainage segment 4a is opened with side holes, and the side holes communicate with the inner chamber. The connection tube segment 4b has a hollow inner chamber. At the connection tube segment 4b, one flushing tube 9 is inserted into the hollow inner chamber, and when approaching the drainage segment 4a, the flushing tube 9 is drawn out of the hollow inner chamber, parallel to the drainage segment 4a, and inserted back into the inner chamber of the drainage segment 4a at a place close to the terminal end of the drainage segment 4a. The flushing tube 9 has via holes on a surface.

Referring to FIG. 7, on an upper wall of the inner chamber of the drainage segment 4a of the diameter-variable tube, there is a sawtooth-shape supporting rib, and sawteeth of the sawtooth-shape supporting rib are oriented towards a tube axis of the diameter-variable tube.

Referring to FIG. 8, the wave-form adhesive-film sealing cover has a corrugated segment, and the corrugated segment has one end connected with a tube-shape connection port, and the other end connected with an outer-edge flap 2b. The tube-shape connection port, the corrugated segment, and the outer-edge flap mentioned above are molded in one piece. The diameter of the wave-form adhesive-film sealing cover is matched with that of the drainage tube 4.

Referring to FIG. 9, the plastic corrugated-tube sealing cover has a corrugated segment, and the corrugated segment has one end connected with a tube-shape connection port, and the other end connected with an outer-edge flap 2b. The outer-edge flap 2b at each side has a first segment and a second segment, and the first segment is perpendicular to the second segment. An end of the corrugated segment away from the tube-shape connection port is connected to a right-angle position formed by the first segment and the second segment, and the first segment is fitted to an inner wall of the corrugated segment.

It is also feasible to use common drainage tubes in the present disclosure, and the above is merely optimal embodiments.

To sum up: the core of the present disclosure is taking advantage of the extendable and retractable effect of the wave-form sealing cover itself to pull out the foam from the deep cavity wound through several operations, thus ensuring the airtightness of the system. Therefore, on the basis of the principle of the present disclosure, all solutions with negative pressure sealing drainage through the wave-form sealing cover which is extendable and retractable fall into the scope of protection of the present disclosure.

1. A negative pressure sealing drainage system for surgical treatment of deep cavity wound, comprising a porous foam, a medical film, a drainage tube, a fluid storage container, and a negative pressure source, wherein the porous foam is a columnar porous foam drainage piece, one end of the drainage tube is inserted into an upper end of the columnar porous foam drainage piece, the columnar porous foam drainage piece is covered by a wave-form sealing cover which is extendable and retractable, an upper end of the wave-form sealing cover is provided with a tube-shape connection port hermetically connected to the drainage tube, a lower end of the wave-form sealing cover is provided with an outer-edge flap, and the medical film covers the outer-edge flap.

2. The negative pressure sealing drainage system for surgical treatment of deep cavity wound according to claim 1, wherein the columnar porous foam drainage piece has a cross-sectional area of 0.8 cm²˜3 cm².

3. The negative pressure sealing drainage system for surgical treatment of deep cavity wound according to claim 1, wherein the columnar porous foam drainage piece has a length of 10 cm˜28 cm.

4. The negative pressure sealing drainage system for surgical treatment of deep cavity wound according to claim 1, wherein the columnar porous foam drainage piece has foam pores.

5. The negative pressure sealing drainage system for surgical treatment of deep cavity wound according to claim 1, wherein a material of the columnar porous foam drainage piece is hydrophilic foam.

6. The negative pressure sealing drainage system for surgical treatment of deep cavity wound according to claim 1, wherein a length of the wave-form sealing cover in a natural state is less than a length of the columnar porous foam drainage piece by 20 mm˜50 mm.

7. (canceled)

8. The negative pressure sealing drainage system for surgical treatment of deep cavity wound according to claim 6, wherein the wave-form sealing cover is a wave-form adhesive-film sealing cover which is extendable and retractable.

9. The negative pressure sealing drainage system for surgical treatment of deep cavity wound according to claim 8, wherein the wave-form adhesive-film sealing cover has a wall thickness of 0.8˜1 mm.

10. (canceled)

11. The negative pressure sealing drainage system for surgical treatment of deep cavity wound according to claim 8, wherein the tube-shape connection port at an upper end of the wave-form adhesive-film sealing cover has a wall thickness of 2 mm˜4 mm.

12. The negative pressure sealing drainage system for surgical treatment of deep cavity wound according to claim 6, wherein the wave-form sealing cover is a plastic corrugated-tube sealing cover which is extendable and retractable.

13. The negative pressure sealing drainage system for surgical treatment of deep cavity wound according to claim 12, wherein the plastic corrugated-tube sealing cover has a wall thickness of 0.3 mm˜0.6 mm.

14. (canceled)

15. The negative pressure sealing drainage system for surgical treatment of deep cavity wound according to claim 12, wherein the tube-shape connection port at an upper end of the plastic corrugated-tube sealing cover has a wall thickness of 1 mm˜3 mm.

16. The negative pressure sealing drainage system for surgical treatment of deep cavity wound according to claim 1, wherein the drainage tube comprises a connection tube segment and a drainage segment interconnected with each other, the connection tube segment is connected to the fluid storage container, an end of the drainage segment away from the connection tube segment is connected to the columnar porous foam drainage piece, the connection tube segment has a hollow inner chamber, the drainage segment has an inner chamber, and the drainage segment is provided with a notch communicating with the inner chamber.

17. (canceled)

18. The negative pressure sealing drainage system for surgical treatment of deep cavity wound according to claim 16, wherein the notch is a groove provided on a tube wall of the drainage segment.

19. The negative pressure sealing drainage system for surgical treatment of deep cavity wound according to claim 18, wherein grooves are in number of four, and the drainage segment is partitioned by the grooves into four portions.

20. The negative pressure sealing drainage system for surgical treatment of deep cavity wound according to claim 19, wherein the inner chamber of the drainage segment is provided with a cross supporting rib for supporting the tube wall of the drainage segment, and four ends of the cross supporting rib are connected to the four portions of the grooves, respectively.

21. The negative pressure sealing drainage system for surgical treatment of deep cavity wound according to claim 16, wherein the drainage tube is a diameter-variable tube, and the diameter-variable segment of the diameter-variable tube has a diameter of 12 mm-24 mm.

22. (canceled)

23. The negative pressure sealing drainage system for surgical treatment of deep cavity wound according to claim 21, wherein the notch is a side hole provided on the tube wall of the drainage segment.

24. The negative pressure sealing drainage system for surgical treatment of deep cavity wound according to claim 23, wherein the inner chamber of the drainage segment is provided with a sawtooth-shape supporting rib for supporting the tube wall of the drainage segment, the sawtooth-shape supporting rib is located on an upper wall of the inner chamber, and tooth tips of the sawtooth-shape supporting rib are oriented towards a tube axis of the diameter-variable tube.

25. The negative pressure sealing drainage system for surgical treatment of deep cavity wound according to any one of claim 16, wherein at the connection tube segment, one flushing tube is inserted into the hollow inner chamber, and when approaching the drainage segment, the flushing tube is drawn out of the hollow inner chamber, and the flushing tube drawn out of the hollow inner chamber runs parallel to the drainage segment.
